(19) United States
(12) Patent Application Publication
(10) Pub. No.: US 2006/0142184 A1
(43) Pub. Date: Jun. 29, 2006

(54) Protein based oral lubricant

(75) Inventor: Dennis James Bannister, Cranebrook, New South Wales (AU)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Pacific Biolink Pty Limited, Hornsby, New South Wales (AU)

(21) Appl. No.: 10/518,615

(22) PCT Filed: Jun. 20, 2003
(86) PCT No.: PCT/AU03/00772

§ 371 Date: Feb. 8, 2006

(30) Foreign Application Priority Data
Jun. 20, 2002 (AU) PS 3032

(51) Int. Cl. A61K 38/16; A61K 31/05

(52) U.S. Cl. 514/7; 514/731

(57) ABSTRACT

The present invention relates to formulations containing phosphoprotein, designed for use as oral lubricant or as artificial saliva, methods of using the formulations and processes for the preparation of the formulations.

### TECHNICAL FIELD

This invention relates to formulations containing phosphoprotein, designed for use as oral lubricant or as artificial saliva, and methods of using the formulations.

### BACKGROUND ART

Phosphoproteins have been described for use in formulations for the treatment or prevention of diseases resulting from the action of bacteria in the oral cavity, for example, caries and gingivitis. However, such formulations have not been described for use as an oral lubricant or as a saliva substitute.

‘Xerostomia’ or ‘Dry Mouth’ is not a disease, but it can be a symptom of certain diseases. Xerostomia can result from medical treatment or as a side effect of many medications. Many times xerostomia is caused by failure of the salivary glands to function normally. Xerostomia can cause health problems by affecting nutrition as well as psychological health. It can contribute to and increase the chances of contracting tooth decay and mouth infections. Causes of Xerostomia include:
a) Medications—Several hundred current medications can cause xerostomia. The major drug groups are antihypertensives and antidepressants. Analgesics, tranquillisers, diuretics, and antihistamines can also cause dry mouth.
b) Cancer Therapy—Chemotherapeutic drugs can change the flow and composition of saliva. Radiation treatment that is focused on or near the salivary gland can temporarily or permanently damage the salivary glands.
c) Systemic diseases—such as Sjogren's syndrome—an autoimmune disease, AIDS, diabetes, and Parkinson's disease.
d) Other conditions—such as bone marrow transplants, endocrine disorders, stress, anxiety, depression, and nutritional deficiencies may cause xerostomia.
e) Nerve Damage—Trauma to the head and neck area from surgery or wounds can damage the nerves that supply sensation to the mouth. While the salivary glands may be left intact, they cannot function normally without the nerves that signal them to produce saliva.
f) Conditions—Alzheimer's disease or stroke may change the ability to perceive oral sensations.

Erosion of tooth structure is defined as the superficial loss of dental hard tissue due to a chemical process not involving bacteria. There can be a variety of chemical factors involved in erosion such as: food, drink, drugs, regurgitation of gastric acid, gastric reflux, and vomiting. Tooth erosion and loss of teeth is common amongst humans or animals with dysfunctional salivary glands and xerostomia. Dysfunctional salivary glands can occur, for example, as a result of radiotherapy in the throat area.

In light of the above it would be advantageous to provide an improved formulation for use as an oral lubricant or saliva substitute to overcome at least some of the disadvantages of known formulations which are unable to provide effective lubrication of the surfaces of the oral cavity.

### DISCLOSURE OF THE INVENTION

The present invention provides formulations for use as an oral lubricant and for use as saliva substitutes in humans or animals, wherein said formulations comprise phosphoproteins. Further, the present invention provides formulations that are particularly useful for treating humans or animals with xerostonia.

An oral lubricant is capable of moistening the mouth and lubricating the surfaces of the oral cavity. An oral lubricant may act in addition to saliva, synergistically with saliva or in the absence of saliva as a saliva substitute. An oral lubricant is particularly useful for patients with substantially or completely defective (or absent) salivary glands and therefore who produce little or no saliva. A saliva stimulant such as pilocarpine stimulates the saliva gland to generate more saliva.

According to a first embodiment, the present invention provides a formulation for the lubrication of the oral cavity in humans or animals, wherein said formulation comprises a water-soluble or water-dispersible form of at least one isolated and purified casein phosphoprotein, or salt thereof.

According to a second embodiment, the present invention provides a formulation for the lubrication of the oral cavity in humans or animals, wherein said formulation comprises a water-soluble or water-dispersible form of at least one isolated and purified casein phosphoprotein, or salt thereof, complexed with at least one bioactive constituent, wherein the bioactive constituent is selected from the group consisting of calcium phosphate and calcium phosphate admixed with at least one other bioactive.

Typically, the formulation may comprise any two or more of the phosphoproteins disclosed herein. Inclusion of casein phosphoprotein in a formulation for lubrication of the oral cavity provides improved lubrication, for example by providing a more effective lining of the surfaces of the oral cavity compared to formulations in which casein phosphoprotein is absent.

Typically, the isolated and purified casein phosphoprotein may comprise at least one casein phosphoprotein as disclosed in Whitney, R. Proteins of Milk. In: Fundamentals of Dairy Chemistry 3^{rd} Edn. (1988) (ed. N. P. Wong), Van Nostrand Reinhold, N.Y., USA, pages: 82-91, the disclosure of which is incorporated herein by reference. More typically, the casein phosphoprotein is selected from the group consisting of: α-casein, β-casein, κ-casein, and mixtures thereof. Yet even more typically, the casein phosphoprotein is selected from the group consisting of:

A. αₛ₁-Caseins

1. αₛ₁-Casein X^{a}-8P (genetic variants-A, B, C, D-9P, and E)

2. αₛ₁-Casein X^{a}-9P (genetic variants-A, B, C, D-10P, and E)

3. αₛ₁-Casein fragments

B. αₛ₂-Caseins

1. αₛ₂-Casein X^{a}-10P (genetic variants-A, B, C-9P, and D-7P)

2. αₛ₂-Casein X^{a}-11P (genetic variants-A, B, C-10P, and D-8P)

3. αₛ₂-Casein X^{a}-12P (genetic variants-A, B, C-11P, and D-9P)

4. αₛ₂-Casein X^{a}-13P (genetic variants-A, B, C-12P, and D-10P)

C. β-Caseins

1. β-Casein X^{a}-5P (genetic variants-A¹, A², A³, B, C-4P, D-4P, and E)

2. β-Casein X^{a}-1P (f 29-209) (genetic variants-A¹, A², A³, and B)

3. β-Casein X^{a}-(f 106-209) (genetic variants-A¹, A³, and B)

4. β-Casein X^{a}-(f 108-209) (genetic variants-A and B)

5. β-Casein X^{a}-4P (f 1-28)^{b}

6. β-Casein X^{a}-5P (f 1-105)^{b}

7. β-Casein X^{a}-5P (f 1-107)^{b}

8. β-Casein X^{a}-1P (f 29-105)^{b}

9. β-Casein X^{a}-1P (f 29-107)^{b}

D. κ-Caseins

1. κ-Casein X^{a}-1P (genetic variants-A and B)

2. Minor κ-Caseins X^{a}-1, -2, -3, etc. (genetic variants-A and B)

Typically, the casein phosphoprotein may be isolated and purified from bovine, ovine or caprine milk. More typically, the casein phosphoprotein for use in the invention may be any commercially available casein phosphoprotein.

Typically, the phosphoprotein within the formulation is designed as an alternative to the protein found in natural saliva.

Typically, the formulation of the present invention may also include a phosphatase inhibitor, More typically, the phosphatase inhibitor is selected from the group consisting of: fluoride ions, anionic polymers, and divalent and trivalent metal ions.

Note that unless otherwise stated, all percentages of components of formulations according to the invention are by weight, based on the total weight of the formulation.

Typically, the amount of phosphoprotein in the formulation is between about 0.0001 and about 60%. More typically, the amount of phosphoprotein present in the formulation is between about 0.01 and 25%. Even more typically, the amount of phosphoprotein in the formulation is between about 0.5 and 20%. More typically still, the amount of phosphoprotein in the formulation is between about 1% and 10%. Preferably, the amount of phosphoprotein in the formulation is between about 2% and 7%. More preferably, the amount of phosphoprotein in the formulation is approximately 3.5%.

Typically, the formulation may also contain calcium phosphate in the form of a complex with the phosphoprotein. Typically, the casein calcium phosphate complex present in the formulation is a discrete chemical entity, and not merely a mixture of phosphoprotein and calcium phosphate as separate species in solution. The complex is understood to exist in the form of a reconstituted, or artificial casein micelle in which amorphous calcium phosphate is stabilised. Typically, the amorphous calcium phosphate is in the form of nanometre-sized particles, hence the aggregate may also be described as nanoclusters.

Typically, the amount of complex in the formulation is between about 0.0001 and about 60%. More typically, the amount of complex present in the formulation is between about 0.01 and 25%. Even more typically, the amount of complex in the formulation is between about 0.5 and 20%. More typically still, the amount of complex in the formulation is between about 1% and 10%. Preferably, the amount of complex in the formulation is between about 2% and 7%. More preferably, the amount of complex in the formulation is approximately 3.5%.

Typically, the amount of calcium present in the formulation is between about 0.0001% and about 25%. More typically, the amount of calcium present in the formulation is between about 0.001% and 0.5%. Even more typically, the amount of calcium present in the formulation is between about 0.002% and 0.1%. More typically still the amount of calcium present in the formulation is between about 0.01% and 0.06%. Preferably the the amount of calcium present in the formulation is about 0.04%.

Typically, the amount of orthophosphate present in the formulation is between about 0.0001% and about 25%. More typically, the amount of phosphate present in the formulation is between about 0.001% and 0.5%. Even more typically, the amount of phosphate present in the formulation is between about 0.002% and 0.2%. Typically still, the amount of phosphate present in the formulation is between about 0.02% and 0.2%. Preferably, the amount of phosphate present in the formulation is between about 0.08% to 0.1%.

Typically, any component that is found in natural saliva may also be a component of the oral lubricant of this invention. Typically, saliva constituents are selected from the group consisting of sodium, potassium, chlorides, fluorides, phosphates, bicarbonates, oxygen, carbon dioxide, urea, enzymes such as ptyalin, maltase and amylase, and proteins such as mucin, globulin, albumen and statherin.

Typically, the pH of the formulation is between about 4.7 and 10. More typically, the pH of the formulation is between about 6 and 8.5. Even more typically, the pH of the formulation is between about 6.5 and 7.5.

Typically, the formulation is a sterile formulation. More typically, the formulation is pasteurised. Suitably pasteurisation substantially eliminates or reduces bacterial contamination that could cause odorous bacterial breakdown or cause illness in the recipient of the formulation.

The functions of the formulation include those of natural saliva; such as:
Wash away food debris and plaque from the teeth to help prevent caries;
Limit the growth of bacteria that cause tooth decay, mouth odor, and other mouth infections;
Bathe the teeth and supply minerals such as calcium and phosphate that allow remineralisation of tooth structure;
Lubricate foods so they may be swallowed more easily;
Moisten the inside of the mouth to make chewing and speaking easier;
Provide enzymes that aid in digestion;
Increase enjoyment of foods by aiding in the “tasting” process;

Typically, the formulation may contain at least one anti-microbial agent. Typically, any commercially available anti-microbial agent may be suitable for use in the formulation of the present invention.

Typically, the anti-microbial agent may be selected from the group consisting of: halogenated diphenyl ethers, such as: 2,4,4-trichloro-2-hydroxy-diphenyl ether (triclosan); phenolic compounds, including phenol and its homologues, such as: 2-methyl-phenol, 3-methyl-phenol, 4-methyl phenol, 4-ethyl phenol, 2,4-dimethyl-phenol, 3,4-dimethyl-phenol, 2,6-dimethyl-phenol, 2,2-methylene bis (4-chloro-6-bromo-phenol); mono- and poly-alkyl and aromatic halophenols, including p-chlorophenols such as: methyl-p-chlorophenol, ethyl-p-chlorophenol, n-propyl-p-chlorophenol, n-butyl-chlorophenol; -o-chlorophenols; p-bromophenols; -o-bromophenols; resorcinol and its derivatives, such as: n-methyl hexyl resorcinol; bisphenolic compounds and halogenated carbanilides and zinc salts.

More typically, the anti-microbial agent may be selected from the group consisting of: glycerol, ethanol, sorbitol, mannitol, sodium benzoate, methyl-p-hydroxy benzoate, ethyl-p-hydroxybenzoate, N-propyl-p-hydroxybenzoate, butyl-p-hydroxybenzoate, phenoxy ethanol and quaternary ammonium salts, such as benzethonium chloride, and diisobutyl-phenoxyethoxyethyl dimethyl benzyl ammonium chloride.

Other types of anti-microbial agents may include amidines, such as substituted guanidine, including chlorhexidine, and other known bis-biguanidines, and cationic tertiary amines.

Typically, the amount of ethanol present in the formulation is between about 0.05 and about 20%. More typically, the amount of ethanol present in the formulation is between about 0.5 and about 10%. Even more typically, the amount of ethanol present in the formulation is between about 1 and about 8%. Yet even more typically, the amount of ethanol present in the formulation is between about 2 and about 6%.

Typically, the antimicrobial agent may be one or more zinc salts.

The formulation of the invention may also be used for the treatment or prevention of mouth odour or halitosis. In a prefered form, the formulation for treatment or prevention of mouth odour or halitosis contains one or more anti-microbials as described herein.

Typically, the formulation of the invention is capable of accepting a suitable amount of a viscosity modifier. Accordingly, the formulation of the invention optionally further comprises at least one viscosity modifier. The viscosity modifier is used to regulate the viscosity of the oral lubricant. Typically, the viscosity of the formulation is similar to that of natural saliva. Alternatively, the formulation of the invention may be a thixotropic gel that liquefies under shear.

The viscosity modifier may be selected from the natural polymers including: proteins, mucin, including synthetic and natural mucin, glycoproteins, hydrolysed proteins, globulin, albumin, statherin, alginate, cellulose and cellulose derivatives. The viscosity modifier may be selected from a synthetic polymer such as: carboxymethyl cellulose.

Other suitable viscosity modifiers include irish moss, gum tragacanth, starch, polyvinylpyrrolidone, hydroxyethylpropylcellulose, hydroxybutyl methyl cellulose, hydroxypropyl methyl cellulose, hydroxy ethyl cellulose (e.g. available as Natrosol), sodium carboxymethyl cellulose, colloidal silica such as finely ground Syloid, laponite (any form of laponite, such as laponite DF), hectorite, calcium montmorillonite, acid activated bleaching earth and palygorskite.

Typically, casein is used as a viscosity modifier.

As the skilled addressee will be aware, the concentration of the viscosity modifier in the formulation will depend on the particular viscosity modifier, or combination of viscosity modifiers, that is present in the formulation. For example, where mucin is used as a viscosity modifier, the mucin is typically present in a concentration between about 0.001% and 20%. More typically, the mucin is present in a concentration between about 0.01% and 10%.

Typically, the formulation of the invention can be used as a mouthwash, and delivered to the oral cavity using any suitable type of vessel, or in the form of a spray. The mouthwash may be swallowed or spat out after application to the oral cavity.

More typically, the formulation is delivered to the oral cavity from a reservoir of artificial saliva, via a tube that is implanted in the body. The reservoir may be stored inside or outside the body. The rate of delivery of the formulation to the oral cavity may be controlled by an electric pump. The pump speed may be selectively adjusted by the operator.

Typically, the rate of delivery of the formulation from the reservoir to the oral cavity is between about 0.01 to about 20 ml/minute. More typically, the rate of delivery of the formulation from the reservoir to the oral cavity is between about 0.05 to about 10 ml/minute. Even more typically, the rate of delivery of the formulation from the reservoir to the oral cavity approximates the natural flow rate of saliva in a healthy individual, which, depending on the physiological status of the individual is typically in the range of about 0.1 to about 10 ml/minute.

Any suitable flavouring or sweetening material may also be employed. Examples of suitable flavouring constituents are flavouring oils. For example, oil of spearmint, peppermint, wintergreen, sassafras, clove, sage, eucalyptus, marjoram, cinnamon, lemon, and orange, and methyl salicylate. Suitable sweetening agents include sucrose, lactose, maltose, dextrose, laevulose, sorbitol, xylitol, d-tryptophan, dihydrochalcones, sodium cyclamate, perillartine, APM (aspartylphenylalanine, methyl ether), saccharine and the like. Suitably, flavouring and sweetening agents may together comprise from about 0.1 to 10% by weight or more of the preparation, and more typically, from about 0.1% to 5% by weight or more of the preparation.

The formulation of the invention optionally further comprises at least one humectant. Humectants contemplated for use in the formulations of the present invention include: glycerol, polyol, sorbitol, polyethylene glycols, propylene glycol, hydrogenated partially hydrolysed polysaccharides and the like. The humectants are generally present in amounts of from 0 to 80%, typically 5 to 70% by weight.

Various other materials may be incorporated in the formulations of the present invention, such as preservatives, silicones, chlorophyll compounds, anti-calculus agents, anti-caries agents, and/or ammoniated material such as urea, diammonium phosphate, and mixtures thereof. These materials including adjuvants, where present, are incorporated in the preparations in amounts which do not substantially adversely affect the properties and characteristics desired.

The formulation disclosed herein may be used for the delivery of any drug that may be taken orally. Typically, the drug may stimulate the production of natural saliva. Typically, the drug designed to stimulate the production of natural saliva may be selected from the group consisting of: Pilocarpine, Salogen and Biotene.

The formulation disclosed herein may be spray-dried to a powder form, for use as a saliva substitute or to supplement the calcium and phosphate levels in the oral cavity of humans or animals with reduced saliva production or having saliva with low concentrations of calcium and phosphate. Typically, the spray-dried formulation is added to confectionary such as chewing gum, lozenges, sweets and the like.

Formulations of the present invention may be incorporated in lozenges, or in chewing gum, sweets or other products, for example by stirring in a pre-dried form of the formulation in solid form into a warm gum based vehicle. Typically, gum based vehicles include jelotone, rubber latex, vinylite resins, and desirably with conventional plasticisers or softeners, sugar or other sweeteners or carbohydrates such as glucose, sorbitol and the like.

More typically, formulations of the invention may be dried, for example, by spray drying, and then added to substances, such as lozenges, chewing gums, carbonated beverages, salt, sugar, artificial sweeteners, baked goods, toothpaste, mouthwash and other oral hygiene products. More typically, formulations of the invention may be included as a liquid formulation, for example as suitable for a mouthwash or spray formulation.

A preferred form of the formulation of the present invention is a chewing gum. The chewing gum may be made from any gum base composition well known in the art and includes those gum bases utilised for conventional chewing gums and bubblegums. Gum bases typically include a polymeric material and may comprise elastomers, resins, polyvinyl acetates, waxes, fats, oils, emulsifiers, fillers and antioxidants.

Typically, a chewing gum in accordance with the present invention may comprise ingredients present in amounts selected from the following ranges: typically, between about 10 to about 80%, more typically, between about 25 to about 80%, even more typically, between about 40 to about 80%, of a gum base; typically, about 0.1 to about 40%, more typically, about 0.1 to about 25%, even more typically, about 1 to about 10% of a pre-dried form of the formulation; typically, between about 5 to about 70%, more typically, between about 10 to about 50%, even more typically, between about 25 to about 40% of a water-soluble bulking ingredient; typically, between 0 to about 5% of a flavourant, more typically, between about 0 to about 3.5% of a flavourant; even more typically, between 0 to about 2% of a flavourant; typically, between 0 to about 0.2%, more typically, between 0 to about 0.1%, even more typically, between 0 to about 0.05% of a colourant; typically, between 0 to about 20%, more typically, between 0 to about 15%, even more typically, between 0 to about 10% of an abrasive; typically, between 0 to about 3%, more typically, between 0 to about 2%, even more typically, between 0 to about 1% of a surfactant; and typically, between 0 to about 3%, more typically, between 0 to about 2%, even more typically, between 0 to about 1% of a fluoridating agent.

As outlined above, unless otherwise stated all percentages of components of the chewing gum form of the formulation are by weight, based on the total weight of the chewing gum composition.

Typically, the chewing gum may be any variety of different chewing gum types including low and high moisture, sugar or sugarless, wax-containing or wax free, low calorie, and the like, and can contain any constituent of the healthy saliva of humans or animals.

In general, a chewing gum product generally consists of a water-insoluble gum base, a water-soluble portion, and flavours. The water-soluble portion dissipates over a period of time, and the gum base portion is retained during mastication. Further, a conventional chewing gum base usually contains an elastomer, an elastomer solvent, and various other ingredients such as fillers, softeners, plasticisers and emulsifiers.

Typically, chewing gum base elastomers include: chicle, jelotong, balata, crown gum, guttapercha, sorva, lechi capsi, nispero, rosidinha, perillo, niger gutta, tunu, gutta kay, pendare, leche de vaca, chiquibul, and the like, butadiene-styrene copolymer, polyisobutylene, isobutylene-isoprene copolymer, polyethylene, and the like, and mixtures thereof. More typically, the amount of elastomers employed in the gum base composition varies greatly depending upon factors such as the type of gum base used, the consistency of the gum base composition desired, and the other components used in the composition to make the final chewing gum product. Even more typically, the elastomer is present in the gum base composition in an amount of between any one of the following: about 15% to about 60%; about 15% to about 30%; or about 25% to about 30%.

Typically, the elastomer solvents are also present in the gum base composition, wherein they act in softening or plasticising the elastomer component. Chewing gum base elastomer solvents include pentaerythritol ester of wood rosin, glycerol ester of polymerised rosin, partially hydrogenated methyl ester of rosin, and the like. More typically, the elastomer solvent may be employed in the gum base composition in an amount of from about 2% to about 40%, and even more typically, from about 7% to about 15%.

Typically, waxes, fats/oils are also present in the gum base composition, wherein they act to improve the elasticity of the gum base. Waxes can provide a soft or firm chew, influence the flavour release and provide bulkiness and smoothness to the gum base. The fats, oils and waxes may be used individually or in combination in the gum base, and may be of mineral, animal, vegetable or synthetic origin. Examples of waxes include paraffin, microcrystalline waxes, polyethylene wax, paraffin wax, beeswax, carnauba wax, microcrystalline wax, candellila wax, rice bran wax, esparto wax, flax wax, sugarcane wax, and synthetic waxes.

Further, examples of suitable oils and fats useable in gum compositions include hydrogenated or partially hydrogenated vegetable or animal fats, and these may be selected from the group consisting of: cottonseed oil, soybean oil, coconut oil, palm kernel oil, beef tallow, hydrogenated tallow, lard, cocoa butter, lanolin and the like, fatty acids such as palmitic, oleic, stearic, linoleic, lauric, myristic, caproic, caprylic, decanoic or esters and salts as sodium stearate and potassium stearate. More typically, these ingredients when used are generally present in amounts up to about 8%, and even more typically up to about 4%.

Generally, the gum base composition may also include effective amounts of fillers or bulking agents, which act to increase firmness and bulk and influence the texture and the flavour release of the chewing gum. Typically, fillers may include organic and inorganic compounds (mineral adjuvants) such as calcium carbonate, magnesium carbonate, ground limestone, magnesium silicate, calcium phosphate, cellulose polymers, clay, alumina, aluminium hydroxide, aluminium silicate, talc, tricalcium phosphate, dicalcium phosphate, and mixtures thereof.

More typically, the amount of the filler present in the gum base composition in an amount from about 1% to about 40%, still more typically, from about 5% to about 20%.

Chewing gum compositions generally include sugar and sugar alcohol sweeteners, having a range in sweetening intensity, which may also act as bulking agents. For example, in sugarless gum compositions, a sweetening agent, such as sorbitol or other sugar alcohol, may act as a bulking agent.

Typically, sugar based sweetening/bulking agents include: monosaccharides, disaccharides and polysaccharides. More typically, the polysaccharides may be selected from the group consisting of: xylose, ribulose, glucose (dextrose), mannose galactose, fructose (levulose), sucrose, maltose, and mixtures thereof. Further, typical sugar alcohol bulking agents include sorbitol, xylitol, mannitol, galactitol, maltitol, mixture of alpha-D-glucopyranosyl-1 6-mannitol and alpha-D-glucopyranosyl-1 6-sorbitol, maltodextrins, hydrogenated starch hydrolysates; hydrogenated hexoses; hydrogenated disaccharides; and the like, and mixtures thereof. Even more typically, the bulking agents/sweeteners may be present in an amount of from about 15% to about 90%, and still more typically, in an amount from about 25% to about 65%, and even more typically, from about 30% to about 50%.

More typically, the chewing gum compositions may also include a high intensity sweetening agent. More typically, the agent is selected from the group consisting of: dihydrochalcone, monellin, steviosides, glycyrrhizin, dihydroflavenol, and L-aminodicarboxylic acid, aminoalkenoic acid ester amides, saccharin and salts thereof, 3,4-dihydro-6-methyl-1,2,3-oxathiazine-4-one-2,2-dioxide and salts thereof, and L-aspartic acid derived sweeteners, such as Aspartame, Alitame, and derivatives of chlorodeoxysucrose or chlorodeoxygalactosucrose.

Typically, the amount of sweetener employed in the chewing gum composition will vary with the sweetener selected for a particular chewing gum and the level of sweetness desired. More typically, the sweeteners are usually present in an amount from about 1% to about 70% and still more typically, in an amount from about 40% to about 50%. Still more typically, the intense sweeteners are usually used in an amount of up to about 1%, and even more typically, from about 0.05% to about 0.4%.

Typically, chewing gum composition may also contain a flavouring agent, and more typically, the flavouring agent is in an amount from about 0.02% to about 5%.

Typically, chewing gum composition may also comprise additives selected from the group consisting of: colouring agents such as: titanium dioxide, incorporated in amounts up to about 2%; thickening agents such as: methyl cellulose, alginates, carrageenan, xanthan gum, gelatin, carob, tragacanth, and locust bean, and fillers.

Typically, in the lozenge according to the present invention, the topical vehicle or carrier in a tablet of lozenge is a solid water-soluble polyhydric alcohol (polyol) such as mannitol, xylotil, sorbitol, malitol, a hydrogenated starch hydrolysate, Lycasin, hydrogenated glucose, hydrogenated disaccharides, and hydrogenated polysaccharides in an amount of about 90 to 98% by weight of the total composition. Solid salts such as sodium bicarbonate, sodium chloride, potassium bicarbonate or potassium chloride may totally or partially replace the polyol carrier.

According to a third embodiment, the present invention provides a method for lubrication of the oral cavity of a human or animal, wherein said method comprises administration to said human or animal of a therapeutically effective amount of a formulation comprising a water-soluble form of at least one isolated and purified casein phosphoprotein, or salt thereof.

According to a fourth embodiment, the present invention provides a method for lubrication of the oral cavity of a human or animal, wherein said method comprises administration to said human or animal of a therapeutically effective amount of a formulation comprising a water-soluble form of at least one isolated and purified casein phosphoprotein, or salt thereof, complexed with at least one bioactive constituent, wherein the bioactive constituent is selected from the group consisting of calcium phosphate and calcium phosphate admixed with at least one other bioactive.

In the method of the third or fourth embodiments, it will be understood that the typical and preferred formulations of the first and second embodiments of the invention may be used.

According to a fifth embodiment, the present invention provides a method for stimulation of saliva production by a human or animal wherein said method comprises administration to said human or animal of a therapeutically effective amount of the formulation of the first or second embodiment together with at least one salivary stimulator.

In accordance with the fifth embodiment the salivary stimulator includes any suitable salivary stimulator such as pilocarpine, salogen, oil of spearmint, peppermint, wintergreen, sassafras, clove, sage, eucalyptus, marjoram, cinnamon, lemon, and orange, and methyl salicylate, sucrose, lactose, maltose, dextrose, laevulose, glycols, sorbitol, xylitol, d-tryptophan, dihydrochalcones, sodium cyclamate, perillartine, APM (aspartylphenylalanine, methyl ether) and saccharine.

According to a sixth embodiment, the present invention provides a method for treating xerostomia in a human or an animal, wherein said method comprises administration to said human or animal of a therapeutically effective amount of the formulation of the first or second embodiment.

According to a seventh embodiment, the present invention provides a method for the treatment and/or prevention of tooth erosion in a human or animal, said method comprising administration to said human or animal a therapeutically effective amount of the formulation according to the first or second embodiment.

Typically, the treatment and/or prevention of tooth erosion involves release of calcium, phosphate and/or fluorophosphate ions from the phosphoprotein and/or phosphoprotein-amorphous calcium phosphate and/or fluorophosphate complexes.

Typically, the prevention of tooth erosion involves the casein phosphoprotein precipitating at pH 4.6 or lower, providing an insoluble lining on the teeth, and therefore preventing release of dental hard tissue.

Typically, in accordance with the first or second embodiment of the invention, the problems associated with xerostomia including tooth erosion, are treated and/or prevented by application of a thixotropic gel, and preferably applied by applying the gel regularly within the oral cavity, thereby applying the formulation to the teeth and gums. Typically, the thixotropic gel formulation is applied to the teeth and gums in a mouth guard for at least two minutes and up to one hour each day, preferably for a duration of two weeks up to 16 weeks or more, and even up to lifetime.

Typically, the formulation is applied in the form of a toothpaste. Typically, abrasives used in the formulations of the present invention, may include alumina and hydrates thereof, such as amorphous silica, alpha alumina trihydrate, magnesium trisilicate, dicalcium phosphate, magnesium carbonate, aluminosilicate, such as calcined aluminium silicate and aluminium silicate, calcium carbonate, zirconium silicate, polymethylmethacrylate, powdered polyethylene, polypropylene, silica xerogels, hydrogels and aerogels and the like. Also suitable as abrasive agents are calcium pyrophosphate, insoluble sodium metaphosphate, calcium metaphosphate, dicalcium orthophosphate, particulate hydroxyapatite and the like. Depending on the form which the oral formulation is to take, the abrasive may be present in an amount of from about 0 to about 70% by weight, typically about 1 to about 70% by weight, more typically from about 10 to about 70% by weight, particularly for toothpastes.

According to an eighth embodiment, the present invention provides a method for providing one or more vitamins and/or minerals to a human or an animal said method comprising administration to said human or animal of a therapeutically effective amount of the formulation of the invention.

Typically, the vitamins may be one or more of any type found in the body of humans or animals. Vitamins may be selected from the group consisting of: Vitamin A, Vitamin C, Vitamin D, Vitamin D₁, Vitamin D₃, Vitamin E, Vitamin K, Vitamin K₁, Vitamin B complex, Vitamin B₁, Vitamin B₂, Vitamin B₆, Vitamin B₁₂, folate, cytamen, and nicotinate.

Typically, the minerals may be one or more selected from the group consisting of: Calcium, phosphate, fluoride, magnesium, barium, strontium, zinc, iron, nickel, aluminium, copper, tin, fluorophosphate, cobalt, sodium, potassium, chloride, bromide, iodide and oxide.

According to a ninth embodiment of the invention there is provided a method for the treatment and/or prevention of mouth odour or halitosis in a human or animal requiring said treatment or prevention, wherein said method comprises administering to the human or animal a therapeutically effective amount of the formulation of the first or second embodiment.

Typically, in the method of the ninth embodiment, the formulation further comprises one or more anti-microbial agents.

According to a tenth embodiment of the present invention there is provided a process for the preparation of an oral lubrication formulation, wherein said process comprises:

(i) adding a water-soluble or water-dispersible form of at least one casein phosphoprotein, and phosphate ions, to an aqueous solution and stirring until the casein is dispersed;

(ii) adding an aqueous solution of calcium ions, with stirring, to the solution formed in part (i), at a pH of 6.5 or greater, and optionally;

(iii) spray drying the product of step (ii).

Typically, in the method of the tenth embodiment, the pH of the solution at, or after, the addition of calcium ions, is between about 6.5 to 10.

According to an eleventh embodiment of the present invention there is provided a process for the preparation of an oral lubrication formulation, wherein said process comprises:

(i) adding a water-soluble or water-dispersible form of at least one casein phosphoprotein to an aqueous solution and stirring until the casein is dispersed; and optionally

(ii) spray drying the product of step (i).

According to a twelfth embodiment of the present invention there is provided use of a formulation according to the first or second embodiment in the manufacture of a medicament for the treatment and/or prevention of xerostomia, tooth decay, mouth odour or halitosis.

### DEFINITIONS

In the context of this specification, the term “comprising” means “including principally, but not necessarily solely”. Furthermore, variations of the word “comprising”, such as “comprise” and “comprises”, have correspondingly varied meanings.

In the context of this specification, the word “phosphate” is used in a manner which includes orthophosphate and fluorophosphate such that the words may herein be used unterchangeably.

In the context of this specification the term “tooth decay” will be understood to include tooth erosion and caries/cavities.

### DESCRIPTION OF PREFERRED EMBODIMENTS OF THE INVENTION

Typically, preferred formulations of the present invention are as follows, wherein all proportions are expressed by weight:

| | |
|---|---|
| Protein | 0.0001 to 60% |
| Water | Up to 100% |

Typically, a preferred formulation of the present invention is as follows:

| | |
|---|---|
| Protein | 0.001 to 20% |
| Calcium | 0.0001 to 5% |
| Phosphate | 0.0001 to 5% |
| Water | Up to 100% |

A more preferred formulation of the present invention is as follows:

| | |
|---|---|
| Protein | 0.01 to 20% |
| Calcium | 0.002 to 0.01% |
| Phosphate | 0.002 to 0.01% |
| Water | Up to 100% |

A further formulation of the present invention is as follows:

| | |
|---|---|
| Protein | 0.01 to 20% |
| Calcium | 0.002 to 0.01% |
| Phosphate | 0.002 to 0.01% |
| Ethanol | 0.05 to 20% |
| Water | Up to 100% |

Typically, a formulation of the present invention may also contain a viscosity modifier as follows:

| | |
|---|---|
| Protein | 0.01 to 20% |
| Calcium | 0.002 to 0.01% |
| Phosphate | 0.002 to 0.01% |
| Mucin | 0.001 to 20% |
| Water | Up to 100% |

Typically, a formulation of the present invention may contain a Vitamin as follows:

| | |
|---|---|
| Protein | 0.01 to 20% |
| Calcium | 0.002 to 0.01% |
| Phosphate | 0.002 to 0.01% |
| Vitamin D | 0.0001 to 0.1% |
| Water | Up to 100% |

Typically, a formulation of the present invention may contain an extra minerals as follows:

| | |
|---|---|
| Protein | 0.01 to 20% |
| Calcium | 0.002 to 0.01% |
| Phosphate | 0.002 to 0.01% |
| Magnesium chloride | 0.0001 to 0.1% |
| Water | Up to 100% |

Typically, a formulation of the present invention may contain an enzyme as follows:

| | |
|---|---|
| Protein | 0.01 to 20% |
| Calcium | 0.002 to 0.01% |
| Phosphate | 0.002 to 0.01% |
| Amylase | 0.00001 to 0.01% |
| Water | Up to 100% |

Typically, a formulation of the present invention may contain a saliva stimulator, as follows:

| | |
|---|---|
| Protein | 0.01 to 20% |
| Calcium | 0.002 to 0.01% |
| Phosphate | 0.002 to 0.01% |
| Ethanol | 0.5 to 20% |
| Pilocarpine | 0.001 to 0.1% |
| Water | Up to 100% |

A specific artificial saliva formulation in accordance with the present invention includes the following components:

| | |
|---|---|
| Protein | 0.5% |
| Disodium phosphate | 0.02% |
| Calcium chloride dihydrate | 0.02% |
| Water | Up to 100% |
| Adjusted to pH 7.0 with 1N NaOH | |

Typically, a formulation of the present invention may be spray-dried to form a powder, including the following components:

| | |
|---|---|
| Protein | 10 to 40% |
| Phosphate | 0.1 to 10% |
| Calcium | 0.1 to 10% |
| Water | Up to 100% |
| Adjusted to pH 7.5 with 1N NaOH | |

Spray-dried formulations can be prepared using standard techniques known to those skilled in the art.

Suitably, a spray-dried powder of the above formulation may be obtained from a Niro Production Minor Spray Drier (Niro Australia Pty. Ltd, Blackburn VIC, Australia), with an inlet temperature of about 200° C., and a flow rate that controlled the outlet temperature to about 85° C.

The resultant spray-dried powder formulation in accordance with the present invention may be then added to vehicles such as: chewing gum, lozenges, foods, beverages, confectionary, pharmaceutical compositions, toothpaste creams or gels, or mouthwashes. The skilled addressee will be aware of appropriate methods for inclusion of the formulation of the invention in a vehicle.

For example, a formulation of the present invention, in the form of a chewing gum may be prepared as follows:

A 2% by weight formulation in accordance with the invention present in the form of a chewing gum comprising: 88% of a gum base, 2% of a spray-dried formulation in accordance with the invention, wherein the spray-dried formulation comprises 4% calcium phosphate and 96% hydrolysed casein; 7% of a water-soluble bulking ingredient; 2% of a flavourant ingredient; and about 0.2% of a colourant; was prepared according to the following.

The gum base may comprise: elastomers, such as crown gum, in an amount of 25 to 55%, elastomer solvents, such as rosin esters, in an amount of 5-25%, waxes, such as paraffin, in an amount of 5-10%, and fillers, such as calcium carbonate in an amount of 15%.

The manner in which the gum base components are admixed is not critical and is performed using standard techniques and apparatus known to those skilled in the art and may be a traditional batch-type process or any extrusion-type process. In a typical method, an elastomer is admixed with an elastomer solvent and/or a plasticiser and/or an emulsifier and agitated for a period of time usually from 1 to 30 minutes. After blending is complete, the remaining ingredients may be added in bulk, incrementally, or stepwise while mixing until a homogeneous mass is obtained. The process may take from 15 minutes to 6 hours in a traditional batch type process. The final mass temperature may vary from 40° C. to 175° C. The final homogeneous mass is discharged from the mixer and allowed to cool and thereafter the gum base composition is incorporated into a chewing gum composition.

The amount of gum base employed in the chewing gum composition will vary depending on such factors as the type of product desired, the type of gum base used, the consistency desired, and the other components used to make the final chewing gum product.

Once prepared, the gum base together with the 2% by weight formulation in accordance with the first embodiment or second embodiment of the invention may be formulated to prepare a wide variety of chewing gum compositions, wherein the chewing gum compositions are prepared using standard techniques and equipment known to those skilled in the art. The apparatus useful in accordance with the present invention comprises mixing and heating apparatus well known in the chewing gum manufacturing arts, and therefore the selection of the specific apparatus would be readily apparent to the person skilled in the art.

Typically, a spray-dried formulation of the present invention may be incorporated into vehicles such as: chewing gum, lozenges, foods, beverages, confectionary, pharmaceutical compositions, toothpaste creams or gels, or mouthwashes, whereby the formulation of the present invention is administered at a rate of between 1 to 100 g per kg of dry weight of the vehicle, such as food. More typically, a spray-dried formulation of the present invention is administered at a rate of between 1 to 75 g per kg of dry weight of food. Even more typically, a spray-dried formulation of the present invention is administered at a rate of between 1 to 50 g per kg of dry weight of food. Yet even more typically, a spray-dried formulation of the present invention is administered at a rate of between 5 and 40 g per kg of dry weight of food. Yet still more typically, a spray-dried formulation of the present invention is administered at a rate of between 5 to 20 g per kg of dry weight of food.

The invention will now be described in greater detail by reference to specific Examples, which should not be construed as limiting on the scope thereof.

### EXAMPLES

The invention will now be described in more detail by way of illustration only, with respect to the following examples. The examples are intended to serve to illustrate the invention and should not be construed as limiting the generality of the disclosure of the description throughout the specification.

Any suitable source of casienate may be used, for example, the sodium caseinate used in the following examples were supplied by New Zealand Milk Products (Australia) Pty. Ltd., 30 Frank St., Wetherill Park, NSW, 2164.

### Example 1

An oral lubricant formulation in accordance with the present invention was prepared according to the following:

Add 0.6% by weight sodium caseinate to 99.2% by weight water, and 0.2% by weight 1N sodium hydroxide, stirring with an overhead stirrer until all the caseinate had dispersed. Then pasteurise.

### Example 2

An oral lubricant formulation in accordance with the present invention was prepared according to the following:

1) Part A: Adding 0.6% by weight sodium caseinate, 0.02% by weight disodium phosphate, to 99.38% by weight water, and 0.2% by weight 1N sodium hydroxide, stirring with an overhead stirrer until all the caseinate had dispersed.

2) Part B: Dissolving 0.4% by weight calcium chloride dihydrate in 99.6% by weight water.

3) Whilst stirring 950 grams of Part A, add dropwise 50 grams of Part B, and continue stirring with an overhead mixer for thirty minutes.

4) Pasteurising.

### Example 3

An oral lubricant formulation containing an anti-microbial constituent in accordance with the present invention was prepared according to the following:

1) Part A: Adding 0.1% by weight phosphoprotein, 0.5% by weight sodium caseinate, 0.04% by weight disodium phosphate, to 5% by weight ethanol, 0.2% by weight 1N sodium hydroxide, and 94.16% by weight water, and stirring until all components had dispersed.

2) Part B: Dissolving 0.8% by weight calcium chloride dihydrate in 99.2% by weight water.

3) Whilst stirring 950 grams of Part A, add dropwise 50 grams of Part B, and continue stirring with an overhead mixer for thirty minutes.

### Example 4

An oral lubricant or artificial saliva formulation containing a viscosity modifier according to the present invention was prepared according to the following:

1) Part A: Adding 0.3% by weight sodium caseinate phosphoprotein, 0.3% by weight hydrolysed casein, 0.02% by weight disodium phosphate, and 0.01% by weight mucin, to 99.17% by weight water and 0.2% by weight 1N sodium hydroxide, and stirring until all components had dispersed.

2) Part B: Dissolving 0.4% by weight calcium chloride dihydrate in 99.6% by weight water.

3) Whilst stirring 950 grams of Part A, add dropwise 50 grams of Part B, and continue stirring with an overhead mixer for thirty minutes.

4) Pasteurising.

### Example 5

An artificial saliva formulation containing a Vitamin according to the present invention was prepared according to the following:

1) Part A: Adding 0.6% by weight phosphoprotein, 0.04% by weight disodium phosphate, and 0.01% Vitamin C, to 0.2% by weight 1N sodium hydroxide and 99.15% by weight water, and stirring until all components had dispersed.

2) Part B: Dissolving 0.8% by weight calcium chloride dihydrate in 99.2% water.

3) Whilst stirring 950 grams of Part A, add dropwise 50 grams of Part B, and continue stirring with an overhead mixer for thirty minutes.

4) Pasteurising.

### Example 6

An artificial saliva formulation containing additional minerals according to the present invention was prepared according to the following:

1) Part A: Adding 0.1% by weight phosphoprotein, 0.5% by weight sodium caseinate, and 0.02% by weight disodium phosphate, to 0 2% by weight 1N sodium hydroxide, and 99.18% by weight water, and stirring until all components had dispersed.

2) Part B: Dissolving 0.4% by weight calcium chloride dihydrate, and 0.2% by weight magnesium chloride in 99.4% by weight water.

3) Whilst stirring 950 grams of Part A, add dropwise 50 grams of Part B, and continue stirring with an overhead mixer for thirty minutes.

4) Pasteurising.

### Example 7

An artificial saliva formulation containing an enzyme according to the present invention was prepared according to the following:

1) Part A: Adding 7.0 grams by weight sodium caseinate, 0.0001% by weight amylase, and 0.02% by weight disodium phosphate, to 1.0% by weight 1N sodium hydroxide, and 91.9799% by weight water, and stirring until all components had dispersed.

2) Part B: Dissolving 0.4% by weight calcium chloride dihydrate in 99.6% by weight water.

3) Whilst stirring 950 grams of Part A, add dropwise 50 grams of Part B, and continue stirring with an overhead mixer for thirty minutes.

4) Pasteurising.

### Example 8

An artificial saliva formulation containing a saliva stimulator according to the present invention was prepared according to the following:

1) Part A: Adding 0.1% by weight phosphoprotein, 0.5% by weight sodium caseinate, 0.04% by weight disodium phosphate, to 5% by weight ethanol, 0.2% by weight 1N sodium hydroxide, 0.1% by weight Pilocarpine, and 94.06% by weight water, and stirring until all components had dispersed.

2) Part B: Dissolving 0.8% by weight calcium chloride dihydrate in 99.2% by weight water.

3) Whilst stirring 950 grams of Part A, add dropwise 50 grams of Part B, and continue stirring with an overhead mixer for thirty minutes.

### Example 9

An artificial saliva formulation that is suitable for delivery to the oral cavity via an implanted tube according to the present invention was prepared according to the following:

1) Part A: Adding 7.0 grams by weight sodium caseinate, 0.0001% by weight amylase, and 0.02% by weight disodium phosphate, to 1.0% by weight 1N sodium hydroxide, and 91.9799% by weight water, and stirring until all components had dispersed.

2) Part B: Dissolving 0.4% by weight calcium chloride dihydrate in 99.6% by weight water.

3) Whilst stirring 950 grams of Part A, add dropwise 50 grams of Part B, and continue stirring with an overhead mixer for thirty minutes.

4) Pasteurising.

### Example 10

A formulation in accordance with the present invention for the use in the treatment and/or prevention of tooth erosion was prepared according to the following:

1) Part A: Adding 0.1% by weight phosphoprotein, 0.5% by weight sodium caseinate, 0.04% by weight disodium phosphate, to 5% by weight ethanol, 0.2% by weight 1N sodium hydroxide, and 94.16% by weight water, and stirring until all components had dispersed.

2) Part B: Dissolving 0.8% by weight calcium chloride dihydrate in 99.2% by weight water.

3) Whilst stirring 950 grams of Part A, add dropwise 50 grams of Part B, and continue stirring with an overhead mixer for thirty minutes.

### Example 11

A spray-dried form of the phosphoprotein-calcium phosphate complex according to the present invention was prepared according to the following:

1) Part A: Adding 23.0% by weight hydrolysed casein, 1.1% by weight disodium phosphate, to 74.4% by weight water and 1.5% by weight 4N sodium hydroxide, and stirring until all components had dispersed.

2) Part B: Dissolving 10.0% by weight calcium chloride dihydrate in 90.0% by weight water.

3) Whilst stirring 950 grams of Part A, add dropwise 50 grams of Part B, and continue stirring with an overhead mixer for thirty minutes.

While the invention has been described in the manner and detail as herein, it will be understood that various omissions, substitutions and/or changes in the form or details described may be made by those skilled in the art without departing from the invention.

1. A formulation for the lubrication of the oral cavity in humans or animals, wherein said formulation comprises a water-soluble or water-dispersible form of at least one isolated and purified casein phosphoprotein, or salt thereof.

2. A formulation for the lubrication of the oral cavity in humans or animals, wherein said formulation comprises, a water-soluble or water-dispersible form of at least one isolated and purified casein phosphoprotein, or salt thereof, complexed with at least one bioactive constituent, wherein the bioactive constituent is selected from the group consisting of calcium phosphate and calcium phosphate admixed with at least one other bioactive.

3. The formulation of claim 1 wherein the casein phosphoprotein is selected from the group consisting of: α-casein, β-casein, κ-casein, and mixtures thereof.

4. The formulation of claim 1 wherein the amount of phosphoprotein present in the formulation is between 0.5 and 20% by weight.

5. The formulation of claim 1 wherein the amount of phosphoprotein present in the formulation is between 2% and 7%.

6. The formulation of claim 2, wherein the amount of casein calcium phosphate complex present in the formulation is between about 0.5 and 20% by weight.

7. The formulation of claim 2, wherein the amount of casein calcium phosphate complex present in the formulation is between about 2% and 7%.

8. The formulation of claim 2, wherein said bioactive constituent is an antimicrobial agent.

9. The formulation of claim 8, wherein the antimicrobial agent is selected from the group consisting of: halogenated diphenyl ethers, such as: 2′,4,4′-trichloro-2-hydroxy-diphenyl ether (Triclosan); phenolic compounds, including phenol and its homologues, such as: 2-methyl-phenol, 3-methyl-phenol, 4-methyl-phenol, 4-ethyl-phenol, 2,4-dimethyl-phenol, 3,4-dimethyl-phenol, 2,6-dimethyl-phenol, 2,2-methylene bis (4-chloro-6-bromo-phenol); mono- and poly-alkyl and aromatic halophenols, including -p-chlorophenols such as: methyl-p-chlorophenol, ethyl-p-chlorophenol, n-propyl-p-chlorophenol, n-butyl-chlorophenol; -o-chlorophenols; p-bromophenols; -o-bromophenols; resorcinol and its derivatives, such as: n-methyl hexyl resorcinol; bisphenolic compounds and halogenated carbanilides.

10. The formulation of claim 9, wherein the antimicrobial agent is selected from the group consisting of: glycerol, ethanol sorbitol, mannitol, sodium benzoate, methyl-p-hydroxybenzoate, ethyl-p-hydroxybenzoate, N-propyl p-hydroxybenzoate, butyl-p-hydroxybenzoate, phenoxy ethanol and quaternary ammonium salts, benzethonium chloride, and diisobutyl-phenoxyethoxyethyl dimethyl benzyl ammonium chloride.

11. The formulation of claim 8, wherein said antimicrobial agent is ethanol and/or one or more zinc salts.

12. The formulation of claim 1, wherein said formulation is incorporated into a formulation selected from the group consisting of: chewing gum, lozenges, foods, beverages, confectionary, pharmaceutical compositions, toothpaste creams or gels, or mouthwashes or spray solutions.

13. The formulation of claim 12 wherein said formulation is incorporated into a chewing gum.

14. The formulation of claim 12 wherein said formulation is incorporated into a lozenge.

15. The formulation of claim 1, wherein said formulation further comprises a viscosity modifier.

16. The formulation of claim 15, wherein said viscosity modifier is selected from the group consisting of: proteins, mucin, including synthetic and natural mucin, glycoproteins, hydrolysed proteins, globulin, albumin, statherin, alginate, cellulose and cellulose derivatives, carboxymethyl cellulose, irish moss, gum tragacanth, starch, polyvinylpyrrolidone, hydroxyethylpropylcellulose, hydroxybutyl methyl cellulose, hydroxypropyl methyl cellulose, hydroxy ethyl cellulose (Natrosol), sodium carboxymethyl cellulose, colloidal silica such as finely ground Syloid, laponite (any form of laponite, such as laponite DF), hectorite, calcium montmorillonite, acid activated bleaching earth and palygorskite.

17. The formulation of claim 15 wherein said viscosity modifier is casein.

18. The formulation of claim 1 wherein said formulation additionally comprises a salivary stimulator.

19. The formulation of claim 18 wherein the salivary stimulator is selected from the group consisting of: pilocarpine and salogen.

20. The formulation of claim 18 wherein said salivary stimulator is selected from the group consisting of: oil of spearmint, peppermint, wintergreen, sassafras, clove, sage, eucalyptus, marjoram, cinnamon, lemon, and orange, and methyl salicylate, sucrose, lactose, maltose, dextrose, laevulose, sorbitol, xylitol, d-tryptophan, dihydrochalcones, sodium cyclamate, perillartine, APM (aspartylphenylalanine, methyl ether) and saccharine.

21. The formulation of claim 1 which is spray-dried prior to use.

22. The formulation of claim 1 which additionally comprises one or more vitamins or minerals.

23. The formulation of claim 22 wherein said vitamin is selected from the group consisting of: Vitamin A, Vitamin C, Vitamin D, Vitamin D₁, Vitamin D₃, Vitamin E, Vitamin K, Vitamin K₁, Vitamin B complex, Vitamin B₁, Vitamin B₂, Vitamin B₆, Vitamin B₁₂, folate, cytamen, and nicotinate.

24. The formulation of claim 22 wherein said mineral is selected from the group consisting of: calcium, phosphate, fluoride, magnesium, barium, strontium, zinc, iron, nickel, aluminium, copper, tin, fluorophosphate, cobalt, sodium, potassium, chloride, bromide, iodide and oxide.

25. A method for lubrication of the oral cavity of a human or animal wherein said method comprises administration to said human or animal of a therapeutically effective amount of a formulation comprising a water-soluble or wafer-dispersible form of at least one isolated and purified casein phosphoprotein, or salt thereof.

26. A method for the lubrication of the oral cavity of a human or animal wherein said method comprises administration to said human or animal of a therapeutically effective amount of a formulation comprising a water-soluble or water-dispersible form of at least one isolated and purified casein phosphoprotein, or salt thereof, complexed with at least one bioactive constituent, wherein the bioactive constituent is selected from the group consisting of calcium phosphate and calcium phosphate admixed with at least one other bioactive.

27. The method according to claim 25 wherein said formulation comprises a water-soluble or water-dispersible form of at least one isolated and purified casein phosphoprotein, or salt thereof.

28. A method for stimulation of saliva production in a human or animal wherein said method comprises administration to said human or animal of a therapeutically effective amount of the formulation of claim 1 together with at least one salivary stimulator.

29. The method of claim 28 wherein the salivary stimulator is selected from the group consisting of: pilocarpine and salogen.

30. The method of claim 28, wherein said salivary stimulator is selected from the group consisting of: oil of spearmint, peppermint, wintergreen, sassafras, clove, sage, eucalyptus, marjoram, cinnamon, lemon, and orange, and methyl salicylate, sucrose, lactose, maltose, dextrose, laevulose, sorbitol, xylitol, d-tryptophan, dihydrochalcones, sodium cyclamate, perillartine, APM (aspartylphenylalanine, methyl ether) and saccharine.

31. A method for treatment and/or prevention of xerostomia in a human or animal wherein said method comprises administration to said human or animal of a therapeutically effective amount of the formulation of claim 1.

32. A method for the treatment and/or prevention of tooth decay in a human or animal, said method comprising administration to said human or animal of a therapeutically effective amount of the formulation according to claim 1.

33. The method according to claim 32, wherein said tooth decay is tooth erosion.

34. A method for providing one or more vitamins and/or minerals to a human or an animal said method comprising administration to said human or animal of a therapeutically effective amount of the formulation of claim 22.

35. A method for the treatment and/or prevention of mouth odour or halitosis in a human or animal requiring said treatment or prevention, wherein said method comprises administering to the human or animal a therapeutically effective amount of the formulation according to claim 1.

36. A method for the treatment and/or prevention of mouth odour or halitosis in a human or animal requiring said treatment or prevention, wherein said method comprises administering to the human or animal a therapeutically effective amount of the formulation according to claim 8.

37. A process for the preparation of an oral lubrication formulation, wherein said process comprises: (i) adding a water-soluble or water-dispersible form of at least one casein phosphoprotein, and phosphate ions, to an aqueous solution and stirring until the casein is dispersed; (ii) adding an aqueous solution of calcium ions, with stirring, to the solution formed in part (i), at a pH of 6.5 or greater, and optionally; (iii) spray drying the product of step (ii).

38. The process according to claim 37, wherein the pH of the solution at, or after, the addition of calcium ions, is between about 6.5 to 10.

39. A process for the preparation of an oral lubrication formulation, wherein said process comprises: (i) adding a water-soluble or water-dispersible form of at least one casein phosphoprotein to an aqueous solution and stirring until the casein is dispersed; and optionally (ii) spray drying the product of step (i).
